(19) United States
(12) Patent Application Publication
(10) Pub. No.: US 2021/0128056 A1
(43) Pub. Date: May 6, 2021

(54) EVALUATION OF TEMPERATURE GRADIENT CHANGES OF TISSUE USING VISUALIZATION

(71) Applicants: Oren Aharon, Haifa (IL); Galit Ankri-Eliahoo, Zichron Ya'akov (IL)

(72) Inventors: Oren Aharon, Haifa (IL); Galit Ankri-Eliahoo, Zichron Ya'akov (IL)

(21) Appl. No.: 16/674,023

(22) Filed: Nov. 5, 2019

(51) Int. Cl. A61B 5/00; A61B 5/026; A61B 5/01

(52) U.S. Cl. CPC A61B 5/444; A61B 5/0261; A61B 5/0077; A61B 2562/0242; A61B 5/449; A61B 5/4312; A61B 5/4387; A61B 5/015

(57) ABSTRACT

This invention relates to apparatus and method for detection of human skin lesion malignancy. The method relies on two independent wavelengths regions: (a) visible and Near IR circa (0.4-1.3 um); (b) Far Infrared (6-12 um). The visible and NIR wavelengths will analyze sub-skin scattering and absorbance to detect structural and diffusion abnormalities, and the FIR wavelengths will monitor the thermal blood perfusion, as reflected by monitoring temperature variation of suspected region. An apparatus according to the present invention would comprise a cooling/heating component which encloses the suspected area, MIR camera or thermal sensors, visible and NIR camera. Evaluation of the rate of the skin temperature changes and propagation in the suspected area will enable to detect increased blood perfusion and metabolic heat, identifying alarming signals related to skin diseases. Parallelly, the sub-skin density and coagulation will be analyzed by peripheral illumination. Evaluation procedure will include image analysis and artificial intelligence to enhance the detection capabilities. Using two independent technologies minimizes the false-negative detection and increases sensitivity and specificity. This invention relates to different body parts without limitations.

### BACKGROUND OF THE INVENTION

### 1. Field of Invention

The field of the invention relates to early detection of alarming signs of skin cancer, and it could be used for screening of nails, breasts and testicles. The proposed apparatus will measure several different parameters to find signs of malignancy. A combination of the two regions of wavelengths increases the available data, offering a better diagnostic tool. There are significant differences between cancerous vs. healthy tissues due to variations in the biochemical composition, blood content, water content, collagen content, density, fiber development and structure. However, there are significant variations from tissue to tissue on one person, in different ages, and between individuals which make it harder to distinguish between healthy and cancerous tissues. Therefore, one of the major obstacles in identifying skin cancer using current electronic devices are False-negative results. By combining several technologies together and identifying different alarming signs the false negative results could be reduces to almost zero.

All biological bodies live in a thermal environment of spatially heterogeneous temperatures. The blood perfusion has great influence on the thermal behavior of skin tissue. The microcirculatory capillaries are located just below the dermis, and small vessels arise almost perpendicularly from it, pass through the dermis, and form a more superficial plexus just below the epidermis. Vascular structures concerned with thermoregulation of the skin consisting primarily of an extensive subcutaneous venous plexus and arteriovenous anastomoses. In a variety of human cancers, blood vessel density was found correlated to tumor aggressiveness and metastasize. Higher levels of micro-vessel densities, as measured in histologic specimen, were found to be associated with adverse prognosis in a variety of tumor. Detection of increased blood perfusion may assist in diagnosis and treatment of malignant lesions and pathologies. The blood perfusion and water content affect the tissue's effective thermal conductivity. Consequently, thermal recovery after cooling of skin surface with increasing blood perfusion was found to be higher and approaches the equilibrium quicker. In this way, it is possible to compare the temperature and the rate of temperature changes of skin tissue in response to external cooling/heating, and identify changes in comparison to nearby areas.

Temperature variation and characterization of biological tissue or material can be evaluated using thermal imaging; for example, high perfusion will yield fast changes at the temperature under external thermal stimulation and shorter time to get to equilibration. The equations for thermal transfer within tissue connect between changes of temperature over time and of mass flow rate and the heat of the blood when it enters the tissue, and the heat generation per unit volume. Creating temperature gradient is performed by cooling or heating the incoming microvessels using thermal ring applied around the lesion. The higher microvessels density in a lesion will create a significant temperature differences from the surrounding which is easily detectable using 8-12 um temperature detecting device. Therefore, it is important to evaluate the temperature characterization of a lesion in comparison to the surrounding skin to identify malignancy. For a specific individual, variation of the temperature between healthy skin tissues vs. suspected lesion, could be detected by observing the temperature changes propagation in parallel to skin layers. Moreover, the information can be saved for future comparisons. It is beneficial to study the expected tissue's temperature characterization: its temperature and rate of change.

The propagation of light under the skin is affected by sub-dermal components like the nucleus. Previous studies showed that light diffusion traveling is different between healthy vs. cancerous tissue in several wavelength ranges. The differences are due to variations in the biochemical composition that occur due to development of malignancy, including larger atypical nuclei and cell volume, increased blood content, water and collagen content, higher density, and atypical structure. These biochemical compositions differences in different tissues affect the absorption and scattering of light traveling. Scattering and absorption of tissues or material can be assessed using imaging; changes at the wave-front of the traveling light can be observed because of high density which may cause high absorption and/or wider angle of light scattering.

To increase the detection probability of alarming signs, there is a need to neutralize natural variations that relate to locations, exposure to sun, age and ethnicity, and focus on parameters relating to malignant tissues. Assessing and differentiating the absorption and scattering properties of a lesion compared to surrounding skin tissue is conducted by a special illumination procedure injected in the periphery area of suspected malignant tissue. Density variation in the biochemical composition of the tissue can be evaluated using specially designed illumination that emphasizes and differentiates between normal and abnormal densities and absorption differences below and above skin. By using a directed beam of light at various wavelengths to travel through the suspected area and observing distortion on its wave-front and auto-fluorescence—a suspicious area could be detected. Moreover, by applying this concept from various directions created by ring of lights on the perimeter of suspected lesion, a comprehensive map could be reconstructed. These phenomena are easily detected using a camera observing the lesion from above.

### 2. Description of Related Art

Skin cancer is the most common cancer in the United States (best available data). In fact, more skin cancers are diagnosed in the US each year than all other cancers combined. For the last three decades, melanoma and skin related problems are growing at an alarming rate worldwide. There are 3 types of skin cancer:
Basal cell carcinoma—This the most common type of skin cancer (˜80%). People who have had basal cell skin cancers are also more likely to get new ones in other places.
Squamous cell carcinoma (˜20%)—Squamous cell cancers are more likely to grow into deeper layers of skin and spread to other parts of the body than basal cell cancers, although this is still uncommon.
Melanoma—melanoma is much less common than basal and squamous cell cancers, but is much more lethal and accounts for the vast majority of skin cancer deaths, and is more likely to grow and spread if left untreated.

Currently the detection of skin cancer is related to external changes: Asymmetry, Border, Color, Diameter and Evolving.

Today, melanoma and skin-related disorders are growing at a fast rate and are regarded as a modern-day epidemic outbreak: More than one out of three new cancers are skin cancer—according to the American Skin Cancer Society about 20% of the population will have skin cancer at least once during their life, and about 2.5% of the population will be diagnosed with a melanoma of the skin, and these rates are constantly rising.

Tumor growth is dependent on angiogenesis, which is process of formation of new blood vessels. Generally, the vessel shape and number stay constant as long as a balance of pro and antiangiogenic stimuli exists. Without angiogenesis, a tumor will not be larger than 1-2 mm and will not metastasize. Higher levels of microvessels densities and increased blood perfusion, were found associated with adverse prognosis in a variety of tumor entities. After a tumor growth, blood perfusion has the main influence on the thermal characterization and behavior of skin tissue. Increase in blood perfusion (as a result of angiogenesis for example), leads to significant increased skin surface temperature, increase in the thermal conductivity of peripheral tissue, as a result it approaches to equilibrium quicker after heating or cooling.

In today technology, skin examination is conducted on the superficial areas by different means, for example: magnifying lens, dermascopes, and various camera related technology which will reveal alarming signs in the external epidermis. However, one of the most important signs of malignancy is increased blood perfusion. Measurement of temperature gradient while applying stable thermal stimulator (e.g. Thermoelectric ring), will provide important information regarding the malignancy of the suspected area/lesion in addition to the structural and biochemical measurements.

Bioheat Transfer Model

Fourier model and non-Fourier heat conduction model describe the relationship between thermal relaxation times and thermal response in tissue.

Fourier heat equation:

q(\vec{r}, ι)=−κ⊥(\vec{r}, ι) (1)

q=heat flex vector=heat flow per unit time per unit area of isothermal surface in the direction of the decreasing temperature; k=thermal conductivity; ∇T=Temperature gradient; r=position vector

The general bioheat transfer equation:

$$ρ c \frac{\partial T}{\partial t} = - ∇ q + {ϖ}_{b} {ρ}_{b} {c}_{b} ( {T}_{a} - T ) + {q}_{m c t} + {q}_{e x t} \qquad ( 2 )$$

ρ=density; c=Specific heat; k=thermal conductivity of the tissue; ρ_{b}, C_{b}=density and Specific heat of the blood; W_{b}=blood perfusion per unit volume; Tₐ, T=Temperatures of blood and tissue, respectively; qₘₑₜ=metabolic heat generation in the tissue; qₑₓₜ=heat generation due to external heat sources.

Since Fourier's Law fails to predict the changes of heat when thermal propagation speed of thermal wave is relatively low. Therefore, an additional parameter tau-q is added to Fourier's low, to compensate the heat wave transfer behavior that is not covered:

$${τ}_{q} = \frac{α}{{C}_{t}^{2}} \qquad ( 3 )$$

τ_{q}=thermal relaxation time; α=thermal diffusivity; Ct=thermal wave's speed in the medium.

q(\vec{r}, ι+τ_{q})=−κν⊥(\vec{r}, ι) (4)

First order Tylor expansion of eq. (4):

$$q ( \vec{r} , t ) + {τ}_{1} \frac{\text{?}}{\partial t} = - \text{?} ( \vec{r} , t ) \\ \text{?} \text{indicates text missing or illegible when filed} \qquad ( 5 )$$

Integration of Eq. (5):

$$q ( \vec{r} , t ) = - \frac{\text{?}}{{τ}_{q}} - exp ( - \frac{\text{?}}{{τ}_{q}} ) \text{?} exp ( \frac{\text{?}}{{τ}_{q}} ) \text{?} \\ \text{?} \text{indicates text missing or illegible when filed} \qquad ( 6 )$$

Eq. (6) presents that the heat flex q(r,t) at time t, depend on the temperature gradient establish from time zero to t. Therefore, it ensures a strong dependency on the thermal gradient. Since tissues are nonhomogeneous, their thermal relaxation time is high and lies in the range of 10-1000 sec.

The thermal wave model of bioheat transfer is conducted from combination of the bioheat transfer eq. and the thermal wave theory:

$${τ}_{q} ρ c \frac{{\partial }^{2} T}{\partial {t}^{2}} = k {∇}^{2} T - {ϖ}_{b} {ρ}_{b} {c}_{b} T - ( {τ}_{q} {ϖ}_{b} {ρ}_{b} {c}_{b} + ρ c ) \frac{\partial T}{\partial t} + ( {ϖ}_{b} {ρ}_{b} {c}_{b} {T}_{b} + {q}_{m} + {q}_{e x t} + {τ}_{q} \frac{\partial {q}_{m}}{\partial t} + τ \frac{\partial {q}_{e x t}}{\partial t} ) \qquad ( 7 )$$

Eq. (7) is also known as a hyperbolic bioheat equation that correlates the temperature gradient with the thermal conductivity of the tissue, its density, blood perfusion, etc. as a function of time.

### SUMMARY OF THE INVENTION

The proposed device includes two different wavelengths regions: visible-NIR and FIR. These technologies will provide different information about the suspected area/lesion. The visible and NIR wavelengths will analyze sub-skin scattering and absorbance of visible and NIR light to detect structural and diffusion abnormalities. A cooling/heating annular device will be applied in the area surrounding the suspected lesion in order to create a temperature-controlled gradient, the FIR wavelengths imaging will detect the temperature gradient as a function of time within the tissues to and detect increased blood perfusion. The results from both technologies will be analyzed and compared, and the system will notify the user if the suspected area will present alarming signs from both technologies.

One of the major differences between malignant and healthy tissue is the formation of new blood vessels angiogenesis. Without angiogenesis a tumor will not be larger than 1-2 mm and will not metastasize. In skin cancer, one of the first stages in the process of benign skin lesion becoming malignant is angiogenesis, before any external changes in the mole/lesion can be detected using naked eye or dermascopes.

The blood circulation plays the main role in changing the skin steady state temperature, compared with the environmental temperature and the metabolic heat generation respectively. Even a small amount of blood changes the temperature significantly from its value when there is no blood flow. Increase in blood flow results in increased temperature, getting to equilibrium after cooling quicker. The metabolic heat generation increased in skin cancer because of the high proliferation of cancerous cells. Although the metabolic heat generation affects the temperature, this effect is uniform and for cool environment with no evaporation, this increase in the metabolic heat generation is negligible when there is a continuous increase in the blood flow rate. It is very important to identify these changes as early as possible to early detect skin cancer even before any noticeable external signs.

Additional difference between malignant and healthy tissue relates to light path within the tissue. Several studies showed that there are significant differences in light diffusion into healthy vs. cancerous skin tissue (melanoma and non-melanoma skin cancer) and depend on the wavelength. The differences are due to variations in the biochemical composition, blood content, water content, collagen content, density, fiber development and structure. In malignant tissues, larger atypical nuclei and larger cell volume are a main cause for the significant increase in the light scattering and variations in the absorption of light. Diffusion properties of biological tissue or material can be evaluated using imaging; for example, wave-front distortion and wider angle of scattering. Skin tissue varies significantly between locations, individuals and depend on age, skin type and ethnicity. Therefore, it is important to evaluate the diffusion properties of lesion in comparison to the surrounding skin.

The proposed art detects the malignancy of tissue/lesion from different aspects: (a) examining the structural changes, by observing distortions in the wave-front of a beam propagating under the skin (b) evaluation of blood flow perfusion within the suspected area by detecting the changes in the temperature and time to equilibrium as observed from above. These detections are based on different densities of malignant lesions and increased blood perfusion and heat generation in malignant tissue, in order to nourish the lesion and provide enough oxygen for intensive cell proliferation process. Following tissue's warm-up after cooling reveals that cancerous tissue warm-up rate is higher than healthy tissue and time to get to equilibrium is shorter; since different rates of temperature changes are directly related to different microvessels densities within the same tissue which may indicate a development of malignant skin lesion.

The above summary does not include an exhaustive list of all aspects of the present invention.

### BRIEF DESCRIPTION OF DRAWINGS

One embodiment of the invention with reference to the appended drawing is now described. Other embodiments of the invention have not been shown in detail as not to obscure the understanding of this description.

FIG. 1 is the skin tissue with malignant lesion and its blood vessels structure

FIG. 2 is a schematic diagram of the preferred embodiment featuring a thermoelectric device (TEC) with central hole, a copper plate below the TEC, a plurality of light sources, and a dual camera for thermal, visible and NIR wavelengths.

FIG. 2A is a transection section of preferred embodiment

FIG. 2B is a longitudinal section of preferred embodiment.

FIG. 3 shows the temperature gradient within the tissue.

FIG. 3A is the temperature gradient in healthy skin tissue.

FIG. 3B is the temperature gradient in cancerous skin tissue.

FIG. 4 shows light wave-front propagation and absorbance within healthy and malignant tissues which is affected by tissue density.

### DETAILED DESCRIPTION OF EMBODIMENTS

The embodiment of the invention here is a system and process that are based on two different wavelengths regions in order to identify several different alarming signs of skin cancer. The first wavelengths region is visible and MR (350-1300 nm)—penetration of light wavelength into the tissue through diffusion. The light sources in the peripheral circle of the device aim to reveal sub-skin abnormalities and evolvement of the mole/ lesion. The light sources are turned-on simultaneously, then in sequential mode, and the captured images of light propagation, absorption and scattering within the tissue from different directions will be saved. For the second wavelength region (FIR 7-12 μm), a thermoelectric cooler will be applied and a copper plate with few millimeters length cylinder at its center will be attached to the thermoelectric cooler. The copper cylinder will form a cooling ring that will be applied on skin surface, and the changes of the temperature propagation and rate will be measured using thermal camera or array of sensors. The plate with the LEDs at wavelengths between 350-1300 nm will be attached to the copper plate to allow the light to penetrate to the skin. The device will contain dual wavelengths camera or two cameras to detect the different wavelengths range.

The device will include a processor to analyze the information that will be received from the dual wavelengths camera or the two cameras: image analysis of sub-skin illumination, light diffusion within the tissue from different directions, analysis of the rate of the temperature changes, and may include processing based on tomographic imaging algorithms.

The techniques described here may have a better capability to distinguish between healthy vs. cancerous tissue and generate a map of the temperature changes and propagation within the tissue to detect changes in blood perfusion, thereby improving cancerous tissue detection.

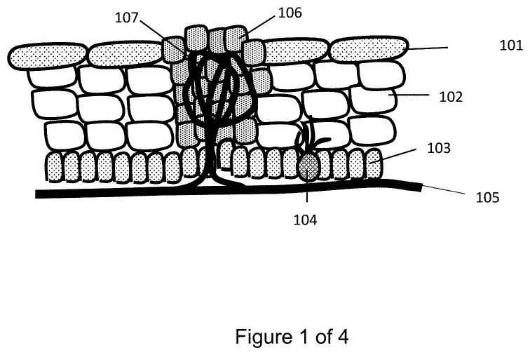

FIG. 1 shows the structure of healthy and malignant skin tissue with malignant lesion and blood vessels development. The upper layer is stratum corneum 101; squamous cells 102; basal cells 103; Melanocytes 104; blood vessels 105; melanoma cells 106; angiogenesis—development of new blood vessels 107. Malignancy is characterized in angiogenesis, applying cold stimuli in the suspected area periphery will accelerate cooling of the malignant tissue by increased heat transfer through blood flow.

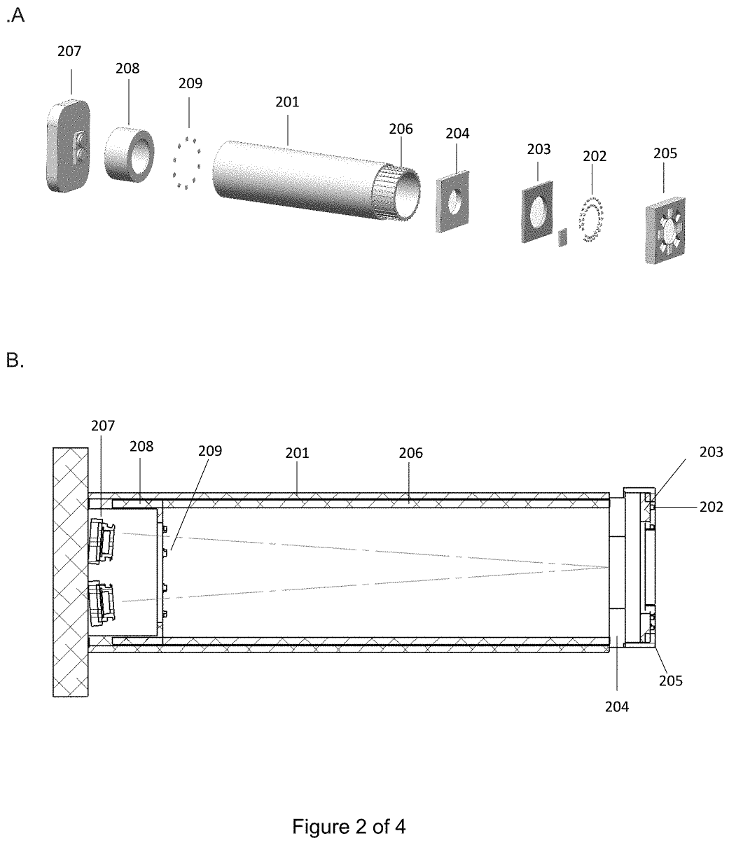

FIG. 2 is an example of an embodiment describing the computerized optical-thermal device that may assist in tissue screening for early detection of cancerous tissue, and it is composed of two different views denoted as 2A and 2B.

FIG. 2A shows the device consists of opaque cylindrical element 201; Light sources 202 are attached to an electric board 203; thermoelectric ring (TEC) 204 is connected on its cold side to copper plate with central hole 205; heat sink 206 are connected to the thermoelectric ring on its warm side along the opaque cylindrical element 201; a dual wavelengths camera 207 is mounted at the central axis of the cylindrical element; Said camera 207 is connected to focus ring 208 therefore it is free to move along its optical axis to allow focusing on various depths; Second set of light sources 209 are connected to the upper-inner side of the opaque cylindrical element 201. The device described in FIG. 2A is applied to the skin surface wherein 205 copper plate touches the tissue and cools the periphery of suspected area. Said light sources 202 illuminate the tissue through machined orifices in 205 members. Said TEC 204 is coupled to 205 on its cold side to actively cool the 205 member, and on its warm side it is equipped with cylindrical heat sink 206. Furthermore, another cylindrical member 201 is used like a spacer to allow mounting cameras designated as 207 and to prevent outside illumination. The dual camera works in two different wavelengths: the first is visible and NIR; second, thermal camera for FIR. The camera is mounted on said 201 cylinder and the light ring is mounted around the optical apertures and illuminated the suspected area from above. Cylinder 208 prevents lights to directly dazzle the camera and creates upper illumination on the suspected area.

FIG. 2B is a cross-section of the embodiment described in FIG. 2A.

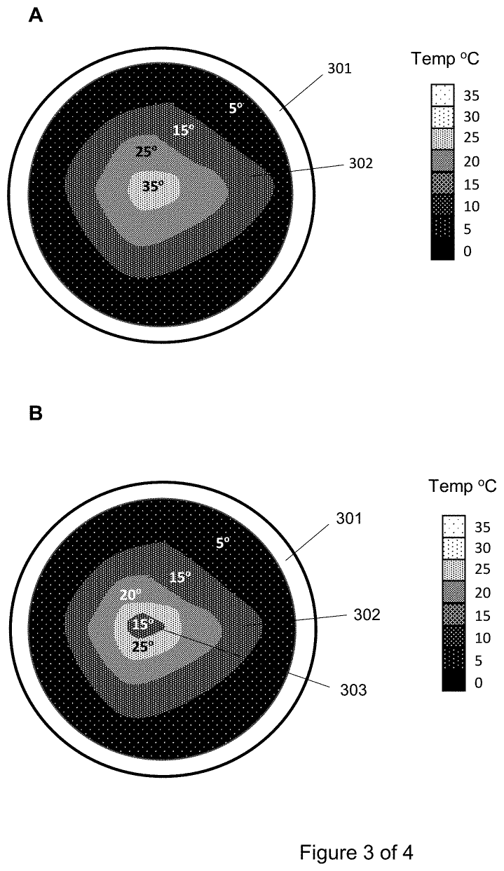

FIG. 3 shows the thermal ring and the temperature gradient within the tissue.

FIG. 3A shows a thermal ring 301 which is applied on healthy tissue 302.

FIG. 3B shows a thermal ring 301 which is applied on a tissue 302 with a malignant lesion 303. The temperature gradient within the healthy tissue is moderate, while the temperature gradient within the cancerous tissue decrease faster and the malignant lesion is cooler than its surrounding tissue, because the blood perfusion to the lesion is significantly higher.

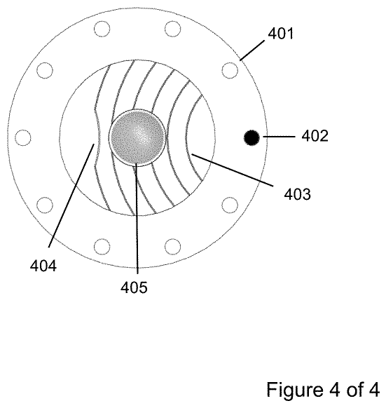

FIG. 4 shows light sources ring 401 with one illuminated light source 402; the wave-front of a beam within the tissue 403 is being distorted 404 by malignant lesion 405. Injecting directed beam of light at various wavelengths into the tissue to travel through by sub-skin diffusion to the suspected area allows to observe wave-front distortion and changes at the absorption and scattering of the light within the suspicious area. Applying this concept from different directions will create a full map of the suspected area. The image created by this phenomenon is recorded by upper camera as described in previous Figures.

Assessing and differentiating the absorption and scattering properties of a lesion compared to surrounding skin tissue is conducted by a special illumination procedure injected in the periphery area of suspected malignant tissue. Density variation in the biochemical composition of the tissue can be evaluated using specially designed illumination that emphasizes and differentiates between normal and abnormal densities and absorption differences below and above skin. By using a directed beam of light at various wavelengths to travel through the suspected area and observing distortion on its wave-front and auto-fluorescence—a suspicious area could be detected. Moreover, by applying this concept from various directions created by ring of lights on the perimeter of suspected lesion, a comprehensive map could be reconstructed. These phenomena are easily detected using a camera observing the lesion from above.

The device will be applied on the suspected tissue area, to create positive attachment between LED source, the cooling ring and the skin tissue. The user will operate the device manually or via computer interface to turn on the thermoelectric ring, light sources simultaneously and in a sequential mode in the peripheral circle enclosing the suspected area. The rate of temperature changes, sub-skin images, and propagation of the light within the tissue and its wave front, light intensity and the angle of light scattering will provide information about different parameters of the preselected area. At the last stage, an analysis algorithm will cross all the information regarding the suspected area and analyze it, in order to find alarming signs for malignancy.

What is claimed is:

1. A medical screening device for skin screening by obtaining blood flow perfusion and diffusion properties of a tissue comprising:
a thermoelectric ring having an orifice on its center placed upon a skin lesion to heat or cool the surrounding of a suspected tissue area;
a temperature detecting device to observe the temperature and temperature gradient as a function of time;
a ring of lights to be applied around a suspect tissue area surface;
an opaque tubular profile to be applied on skin surface concentric to said ring of lights allow light to penetrate only through the tissue;
said ring of lights comprising of multiple sources of light in wavelengths 0.3-1.3 um, each one controllable independently;
one or more cameras sensitive to said light emitting sources; and
a computer vision device to analyze and compare information from said cameras and temperature detecting device.

2. The device of claim 1 wherein the temperature detecting device is a thermal camera.

3. The device of claim 1 further compromising a camera capable of shifting its focus for different depths within the tissue.

4. The device of claim 1 wherein said tissue includes: skin, nail, breast, testicle, and the measured properties are related to density, absorbance and scattering, blood content and heat generation of the tissue.

5. A method and apparatus for measuring the diffusion properties and blood perfusion of a skin tissue comprising:
activating a thermoelectric ring having an orifice placed upon a skin lesion to heat or cool the surrounding of a suspected tissue area;
recording images or data from a temperature detecting device to observe the temperature and temperature gradient including as a function of time;
activating a ring of light sources with wavelengths range between 300-1300 nm, around a lesion, each light source controlled independently;
recording images created by each said light source as it propagates by diffusion under the skin; and
analyzing the light propagation wavefront for detecting irregularities.

6. The method and apparatus of claim 5 wherein the temperature detecting device is a thermal camera.

7. The method and apparatus of claim 5 wherein the pictures are taken from different depths within the tissue.

8. The method and apparatus of claim 5 wherein said tissue includes: skin, nail, breast, testicle, and the measured properties are related to density, absorbance and scattering, blood content and heat generation of the tissue.
